(19) United States
(12) Patent Application Publication
(10) Pub. No.: US 2020/0188272 A1
(43) Pub. Date: Jun. 18, 2020

(54) COSMETIC COMPOSITION HAVING COTTON CANDY FORM

(71) Applicant: TEGO SCIENCE INC., Seoul (KR)

(72) Inventors: Saewha JEON, Seoul (KR); Yun Hee KIM, Seoul (KR); Jikhyon HAN, Seoul (KR)

(73) Assignee: TEGO SCIENCE INC., Seoul (KR)

(21) Appl. No.: 16/483,747

(22) PCT Filed: Nov. 22, 2017
(86) PCT No.: PCT/KR2017/013336

§ 371 Date: Aug. 6, 2019

(30) Foreign Application Priority Data
Feb. 8, 2017 (KR) 10-2017-0017669

(51) Int. Cl. A61K 8/73; A61K 8/02

(52) U.S. Cl. CPC A61K 8/733; A61K 8/0216

(57) ABSTRACT

The present invention relates to a cosmetic composition having a cotton candy form. The cosmetic composition in a cotton candy form is prepared by freeze-drying an algin aqueous solution, and thus can be prepared through a simple process, has excellent moldability, and can stably contain, even at room temperature, a protein such as a cytokine, a peptide, and the like, which have particular storage requirements.

### TECHNICAL FIELD

The present invention relates to a cosmetic composition in the form of cotton candy.

### BACKGROUND ART

Recently, a lot of sheet masks or patch cosmetics have been released in the field of cosmetics. These types of products are prefabricated with specific designs such as a facial shape, a spot, and the like, so they can be attached to the skin while fitting the shape without needing to be applied by hand. These products have entered the spotlight as a product capable of more effectively obtaining cosmetic effects such as moisturizing, cleansing, whitening, wrinkle improvement, and the like compared to general skincare products.

The types of sheet masks or patch cosmetics include wet-type masks in which a non-woven fabric and the like utilized as a support is soaked with cosmetic essences, hydrogel masks fabricated by hardening cosmetic essences with a gelling agent, and the like. However, since it is difficult for these types of masks to carry water-unstable active ingredients and there is little change before and after use, they are less effective and interesting.

Therefore, in recent years, a melting formulation has appeared in which the active ingredient is stably stored in an anhydrous formulation and is completely dissolved on the skin when in contact with water-containing essences, serums, and the like. A representative form of the melting formulation is a water-soluble film fabricated by drying a water-soluble film-forming agent such as starch, cellulose, pullulan, and the like, but this has many disadvantages such as a large amount of residue remaining on the skin, stickiness resulting from the dissolution of a film-forming agent, skin tightening resulting from the formation of a thin film after drying, and the like.

Another form of the melting formulation is a patch fabricated by a freeze-drying method, and the patch is known to be in the form of a sponge made of hyaluronic acid, collagen, or a combination thereof. This sponge patch exhibits a significant difference in solubility according to a thickness thereof. A thin patch is easily dissolved, but a patch with a thickness of 1 mm or more has a problem of solubility even though it attains satisfactory strength. In addition, a sponge patch form fabricated by freeze-drying hyaluronic acid exhibits very low solubility in water and becomes extremely sticky when used in a dissolved state in an aqueous emulsion, and a sponge patch form fabricated by freeze-drying collagen is not completely melted.

Accordingly, there is a need for research and development on a novel cosmetic formulation having high solubility and capable of stably containing a protein.

### REFERENCE DOCUMENTS

1. Korean Registered Patent No. 10-1103265

### DISCLOSURE

### Technical Problem

The present invention is directed to providing a cosmetic composition in the form of cotton candy, which stably contains a protein that requires a particular storage condition.

### Technical Solution

To address such a challenge, the inventors of the present invention have found that a cosmetic composition in the form of cotton candy, which is prepared by freeze-drying an aqueous algin solution, has excellent solubility and stably retains a protein even at room temperature, and the present invention has been completed based on these facts. One aspect of the present invention provides a cosmetic composition in the form of cotton candy, which is prepared by freeze-drying a 0.03 to 10% (v/v) aqueous algin solution and has a Shore hardness of at most 150 and a dissolution rate in a solvent of 0.01 to 10 mg/sec.

Another aspect of the present invention provides a cosmetic kit which includes: the above-described cosmetic composition in the form of cotton candy; and a liquid cosmetic composition for applying the cotton candy onto the skin.

### Advantageous Effects

A cosmetic composition in the form of cotton candy according to the present invention can be prepared by a simple process, that is, by freeze-drying an aqueous algin solution, and can also stably contain a protein or peptide such as a cytokine, and the like, which requires a particular storage condition.

### DESCRIPTION OF DRAWINGS

FIG. 1 shows images of the form of cotton candy prepared in varying preparation containers.

FIG. 2 shows images of the form of cotton candy prepared at varying concentrations.

FIG. 3 shows images illustrating the restorability of the cotton candy form according to the present invention by showing the cotton candy before (left) and after (right) a constant force is applied.

FIG. 4 shows images (time sequence) of sequentially observing the rate at which cotton candy is dissolved in a solvent when mixed with a second solution.

FIG. 5 shows a western blot result for determining the protein stability of protein-containing cotton candy (Lane 1 (L1): no protein added, L2: 0 month, L3: 1 month, L4: 3 months, L5: 5 months, L6: 7 months, L7: 9 months, and L8: 12 months).

### BEST MODE

The benefits and features of the present invention, and the methods of achieving the benefits and features will become apparent with reference to exemplary embodiments to be described below in detail along with the accompanying drawings. However, the present invention is not limited to the exemplary embodiments to be disclosed below and may be implemented in various other forms, and the present exemplary embodiments are provided for rendering the disclosure of the present invention complete and for fully conveying the scope of the invention to a person with ordinary skill in the technical field to which the present invention pertains, and the present invention will be defined only by the scope of the claims.

### <Example 1> Preparation of Cosmetic Composition in Form of Cotton Candy

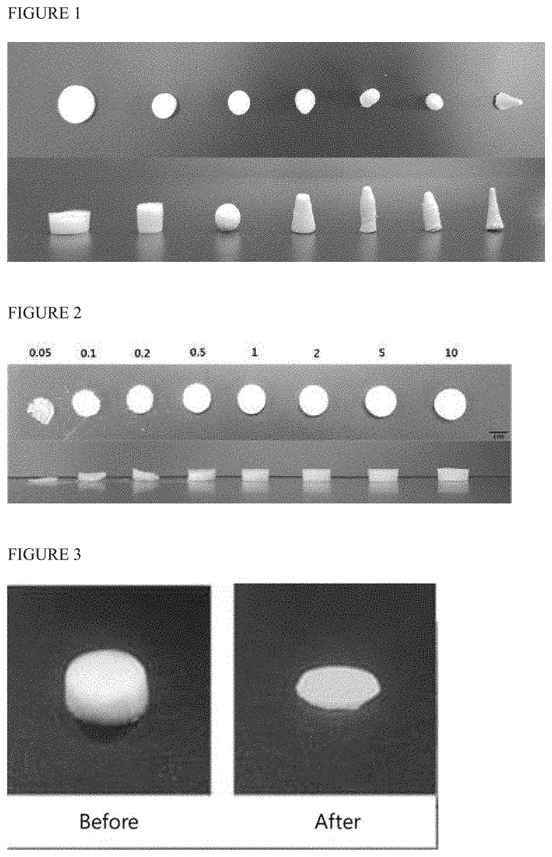

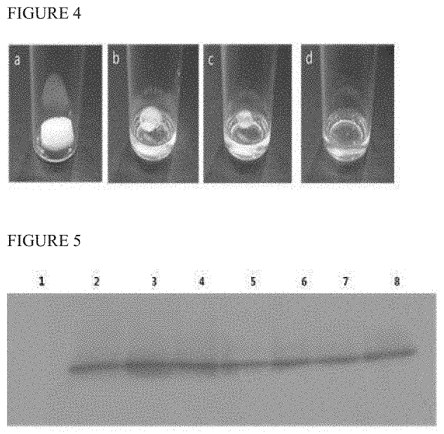

Algin was dissolved in sterile distilled water at varying concentrations of 0.01%, 0.02%, 0.05%, 0.1%, 0.2%, 0.5%, 1%, 2%, 5%, and 10% to prepare aqueous algin solutions. Each of the prepared aqueous algin solution was put into a container, frozen at −40° C., and then dried at 25° C. for about 16 hours to prepare a composition in the form of cotton candy (see FIGS. 1 and 2).

As a result, it can be seen that the form of the composition is stably prepared at 0.05% or more, but the form thereof is not prepared at 0.02% and 0.01%. In addition, it can be seen that, when a constant force is applied, the form of the composition is not restored unlike characteristics of a general sponge form, indicating that it has the same characteristic as that of cotton candy (see FIG. 3).

### <Example 2> Confirmation of Physical Properties (Density, Hardness, and Dissolution Rate) of Cotton Candy

Each cotton candy prepared by freeze-drying at varying concentrations can be mixed with a second solution and then applied onto the skin. Therefore, in order to confirm the physical properties of cotton candy prepared at varying concentrations in Example 1, cylindrical cotton candy among several forms of cotton candy shown in FIG. 1 was used to measure density, hardness, and a dissolution rate at varying concentrations. The hardness of the cotton candy form was measured using a Shore hardness gauge in accordance with ASTM D 2240.

To measure the rate at which cotton candy was dissolved in a solvent, distilled water or glycerol was used. The solubility in glycerol was measured separately for 1% glycerol and 10% glycerol. The measurement of solubility was performed by measuring an elapsed time until cotton candy is completely dissolved from the moment one piece of cotton candy is in contact with each of five types of solutions at room temperature without any physical action such as stirring or shaking, and results thereof were obtained as shown in Table 1.

TABLE 1

| Concentration | | | Dissolution rate of cotton candy | | |
|---|---|---|---|---|---|
| of aqueous | | | in 5 ml of solvent (mg/sec) | | |
| algin solution | Density | Hardness | Distilled | Glycerol | Glycerol |
| (%) | (mg/cm³) | (Shore D) | water | 1% | 10% |
| 0.01 | — | — | — | — | — |
| 0.02 | — | — | — | — | — |
| 0.05 | 1.132 | 0.2 | 0.100~1.600 | 0.100~1.600 | 0.100~1.600 |
| 0.1 | 1.510 | 1 | 0.100~1.600 | 0.100~1.600 | 0.020~0.320 |
| 0.2 | 3.114 | 2 | 0.110~1.600 | 0.092~1.333 | 0.028~0.040 |
| 0.5 | 5.322 | 3 | 0.039~0.667 | 0.039~0.667 | 0.026~0.444 |
| 1 | 11.210 | 7 | 0.062~1.000 | 0.058~0.930 | 0.041~0.667 |
| 2 | 21.288 | 20 | 0.085~1.364 | 0.084~1.339 | 0.067~1.071 |
| 5 | 50.955 | 55 | 0.078~1.241 | 0.073~1.169 | 0.058~0.923 |
| 10 | 94.324 | 79 | 0.038~0.582 | 0.037~0.571 | 0.023~0.356 |

### <Example 3> Confirmation of Protein Stability of Protein-Containing Cotton Candy

After storing cotton candy containing a protein at room temperature for as much as 12 months, the protein was extracted, and protein stability was analyzed through western blotting. The protein extracted from the cotton candy was subjected to electrophoresis in a SDS-PAGE gel and then subjected to western blotting through a method widely known in the art.

As a result, it can be seen that the protein contained in cotton candy is stably retained at room temperature until 12 months (see FIG. 5).

Modes of the Invention

Hereinafter, the present invention will be described in detail.

The inventors of the present invention have studied a cosmetic composition that stably retains a functional substance such as a protein or a peptide, and as a result, a cosmetic composition in the form of cotton candy was obtained by molding and freeze-drying an aqueous algin solution. The cosmetic composition in the form of cotton candy can be prepared by a simple process so as to have properties suitable for being used as a cosmetic composition, that is, a density of 0.1 to 200 mg/cm³, a Shore hardness of at most 150, and a dissolution rate in 5 ml of a solvent of 0.01 to 10 mg/sec, and can also stably retain a functional substance such as a protein or a peptide.

The present invention provides a cosmetic composition in the form of cotton candy, which is prepared by freeze-drying a 0.03 to 10% (v/v) aqueous algin solution and has a Shore hardness of at most 150 and a dissolution rate in a solvent of 0.01 to 10 mg/sec.

As used herein, the term “form of cotton candy (fairyfloss or candyfloss)” means a form of cotton candy prepared through freeze drying. The form of cotton candy is soft like cotton candy, has a large surface area due to its porosity, and is completely dissolved immediately when in contact with a solution due to its high solubility. In addition, when a constant force is applied, the form is not restored.

According to an embodiment of the present invention, in order to prepare a cosmetic composition in the form of cotton candy, algin is mixed with sterile distilled water to prepare aqueous algin solutions at varying concentrations, and each of the aqueous algin solutions is put into a freezing container and freeze-dried to prepare a form of cotton candy. According to an embodiment of the present invention, the algin may be alginic acid or an alkali metal alginate, such as glyceryl alginate, diosgenin alginate, methylsilanol carboxymethyl theophylline alginate, sodium alginate sulfate, siloxanetriol alginate, ethyl lauroyl arginate HCl, calcium alginate, TEA-alginate, potassium alginate, propylene glycol alginate, PCA ethyl cocoyl arginate, ammonium alginate, magnesium alginate, potassium undecylenoyl alginate, silanetriol arginate, sodium/TEA-undecylenoyl alginate, ethyl lauroyl arginate laurate, hydrolyzed algin, or the like.

Particularly, sodium alginate, potassium alginate, or ammonium alginate, which is highly water-soluble, is preferred.

According to an embodiment of the present invention, the concentration of algin contained in the aqueous algin solution may be, but the present invention is not limited to, 0.03 to 10% (v/v).

According to an embodiment of the present invention, the aqueous algin solution may be frozen at −5° C. to −70° C. and then dried at 15° C. to 30° C. for about 10 hours or more, and the freeze drying may be performed by a method known in the art.

According to an embodiment of the present invention, the cosmetic composition in the form of cotton candy may have a density of, but the present invention is not limited to, 0.1 to 200 mg/cm³.

According to an embodiment of the present invention, the cosmetic composition in the form of cotton candy may have a Shore hardness of, but the present invention is not limited to, at most 150. In addition, regarding the restorability of the form after pressure is applied, the form of the cosmetic composition may not be restored.

According to an embodiment of the present invention, the cosmetic composition in the form of cotton candy may have a dissolution rate in 5 ml of a solvent of, but the present invention is not limited to, 0.01 to 10 mg/sec.

The solvent may be one or more selected from the group consisting of purified water, saline, and glycerol.

When the solvent is purified water, a dissolution rate of the cosmetic composition in 5 ml of purified water may be 0.1 to 10 mg/sec, more preferably, 0.1 to 2 mg/sec, but the present invention is not necessarily limited thereto.

When the solvent is glycerol, a dissolution rate of the cosmetic composition in glycerol may be 0.1 to 10 mg/sec, more preferably, 0.1 to 2 mg/sec, but the present invention is not necessarily limited thereto.

In an example, when the solvent is glycerol and the concentration of glycerol is 1%, a dissolution rate of the cosmetic composition in 1% glycerol may be 0.1 to 10 mg/sec, more preferably, 0.1 to 2 mg/sec, but the present invention is not necessarily limited thereto.

In another example, when the solvent is glycerol and the concentration of glycerol is 10%, a dissolution rate of the cosmetic composition in 10% glycerol may be 0.1 to 10 mg/sec, more preferably, 0.1 to 2 mg/sec, but the present invention is not necessarily limited thereto.

In addition, the cosmetic composition in the form of cotton candy may further include a functional substance.

According to an embodiment of the present invention, the functional substance may be one or more selected from the group consisting of a liquid culture, a cell extract, a cytokine, a chemokine, a growth factor, a peptide, a natural extract, a compound, a chemical substance, a synthetic polymer, a biopolymer, deoxyribonucleic acid (DNA), ribonucleic acid (RNA), and the like, but the present invention is not limited thereto.

The functional substance may include any one or a mixture of two or more of a skin whitening ingredient, a skin wrinkle improving ingredient, an UV blocking ingredient, an anti-oxidation ingredient, a skin/hair conditioning ingredient, and an antimicrobial ingredient, but the present invention is not limited thereto.

In an embodiment of the present invention, the protein stability of cotton candy prepared by freeze-drying varying concentrations of an aqueous algin solution was determined. As a result, it can be seen that a protein contained in the cotton candy is stably retained at room temperature until 12 months.

Therefore, the cosmetic composition in the form of cotton candy according to the present invention, which may further include a functional substance, can stably contain and retain the functional substance at room temperature for a long period of time.

In addition, since the cosmetic composition in the form of cotton candy according to the present invention can be prepared into various shapes such as a cylindrical shape, a spherical shape, a conical shape, a polyhedron, and the like according to a preparation container, very high moldability is exhibited (see FIG. 1).

In addition, the present invention provides a cosmetic kit which includes the above-described cosmetic composition in the form of cotton candy and a liquid cosmetic composition for applying the cotton candy onto the skin.

1. A cosmetic composition in the form of cotton candy, which is prepared by freeze-drying a 0.03 to 10% (v/v) aqueous algin solution and has a Shore hardness of at most 150 and a dissolution rate in a solvent of 0.01 to 10 mg/sec.

2. The cosmetic composition of claim 1, which has a density of 0.1 to 200 mg/cm³.

3. The cosmetic composition of claim 1, wherein the solvent is one or more selected from the group consisting of purified water, saline, and glycerol.

4. The cosmetic composition of claim 1, which further comprises a functional substance.

5. The cosmetic composition of claim 4, wherein the functional substance is one or more selected from the group consisting of a liquid culture, a cell extract, a cytokine, a chemokine, a growth factor, a peptide, a natural extract, a compound, a chemical substance, a synthetic polymer, a biopolymer, deoxyribonucleic acid, and ribonucleic acid.

6. The cosmetic composition of claim 4, which stably retains the functional substance for a long period of time, even at room temperature.

7. A cosmetic kit comprising:
the cosmetic composition in the form of cotton candy of claim 1; and
a liquid cosmetic composition for applying the cotton candy onto the skin.
